(19) United States
(12) Patent Application Publication
(10) Pub. No.: US 2022/0369601 A1
(43) Pub. Date: Nov. 24, 2022

(54) BREEDING COMB FOR SOLITARY BEES

(71) Applicants: Damian KAPKA, Glowno (PL); Przemyslaw KAPKA, Glowno (PL)

(72) Inventors: Damian KAPKA, Glowno (PL); Przemyslaw KAPKA, Glowno (PL)

(21) Appl. No.: 17/627,123

(22) PCT Filed: Jul. 10, 2020
(86) PCT No.: PCT/PL2020/000062

§ 371 Date: Jan. 13, 2022

(30) Foreign Application Priority Data
Jul. 13, 2019 (PL) P.430579

(51) Int. Cl. A01K 47/04

(52) U.S. Cl. CPC A01K 47/04

(57) ABSTRACT

Breeding comb for solitary bees is comprised of nesting sheets stacked one on top of another and containing grooves forming nesting tunnels (1) of varying sizes, lugs (2) placed over the side edges of the sheet and on the back wall, locks in the form of lugs (4) in the lower part of the base of the nesting sheet forming pass-through channels (5), slide guides (6) placed in the front part of the sheet, interfaces (8) placed on outer sides of the sheets where the individual nesting sheets are separated from each other by a membrane (3), with a shutter (7) placed at the front part of the breeding comb containing inlet holes (9) of varying sizes corresponding to the sizes of the nesting tunnels (1), and holes (10) corresponding to the sizes of the pass-through channels (5).

The present invention relates to a breeding comb to be used in the field of beekeeping for breeding solitary bees.

In the general opinion, bees are above all producers of honey and wax as well as the other bee products. Meanwhile, the most important role of bees for humans is the pollination of cultivated and wild plants.

Progressive specialisation in horticulture, decreasing number of apiaries with honeybees, as well as the chemicalisation of agriculture, cause the collapse of honeybee colonies and make the problem of crop pollination more and more important for the stabilisation of yields.

It is estimated that bees from breeding colonies cover approximately half of the needs concerning pollination of plants. The remaining gap can be potentially filled by wild pollinating insects, primarily bumblebees and solitary bees, but their population is unfortunately constantly decreasing. The apidae are characterised by great species diversity. The genera and species distinguished among solitary bees include Yellow-faced Bee (Hylaeus), Small Carpenter Bee (Ceratina), Heriades, Small Scissor Bee (Chelostoma), Mason bees (Hoplitis, Osmia), Banded Mud Bee (Chalicodoma), Wood-carving Leaf-cutter (Megachile) and Wool Carder Bee (Anthidium). In natural conditions, bees use as nests places like hollow plant stems, e.g. reeds, as well as other cavities of other kinds.

The great adaptability of some species is evidenced by the fact that they can nest in very unusual places, e.g. hollow bricks or thatched roofs. Their nests are of a linear nature; they consist of successively arranged larval cells. Some species form colonies of nests thriving under conditions of easy access to a large number of channels. The gathering of pollen into the nests begins from the end of the channels, which are blanked off, and progresses towards the entrance, Each single cell is filled with pollen by the females and then it is equipped with an egg and separated from the next cell with a species-specific barrier. Depending on the length of the channel, typically several to more than a dozen larval cells are formed in the nest. After sealing the last cell, the entrance to the nest is secured with an appropriate plug that protects the larvae against natural enemies and unfavourable weather conditions.

Solitary bees search for food within a small distance from the nests (approx. 700 m) and they feed on a wide range of foods. Especially remarkable is the complete lack of aggressiveness towards people, which makes it possible to use solitary bees in kitchen gardens.

The traditional rearing of solitary bees makes use of common reed for nests. Disadvantages of the commonly used nesting materials include the greatly labour-consuming preparation of reed nests (i.e. the annually repeated harvesting, selection, defoliation and cutting of reed stems), the risk of allergic sensitisation (when the reeds are torn open and the cocoons are taken out, tiny particles of unused pollen and spores of pathogenic fungi are released to the air), the unfeasibility of removing the remains of cocoons from the nest and carrying out a selection thereof as well as the difficulties with getting completely rid of pests and parasites.

From the description PCT/IB2007/001989, published as WO2007/122513, there are known solutions relating to a method and an apparatus for breeding and to an incubator for solitary bees, which rely on the use of corrugated sheets which, when folded in half, form nesting tunnels. In another embodiment, the corrugated sheets can be made separately. Nesting tunnels are formed in such manner that when two sheets are assembled together, the recesses of one sheet are compatible with the recesses of the other plate and thus make nesting tunnels in a form similar to hexagons. Therefore, in order for the upper and lower sheets to be properly assembled in the solution according to this invention, the recesses in both sheets must be of identical size, which in result produces nesting tunnels of equal size.

A disadvantage of such a solution is that it is not possible to differentiate the size of nesting tunnels in one hive. Solitary bees have different sizes and observations of these creatures indicate that they prefer to inhabit nesting tunnels similar to the size of the bee itself.

Therefore, in order to adapt the nesting tunnels to each type of bee, it would be necessary to set up several different hives.

From the utility model description PL67276, there is known a device in which thermoformable foil is used for the production of nest sheets. Unfortunately, there is a barrier in this method related to the thickness of the walk of the sheets. They are thinner and thus have shorter lifetime. Furthermore, the sheets made according to the utility model referred to above have different thickness at different places and are easily damaged mechanically when the cocoons are removed. Moreover, these sheets can become deformed when exposed to higher temperatures, e.g. under exposure to solar radiation.

From the description of utility model PL 124947, there is known a device in which the breeding comb is comprised of unit modules in the form of a sheets containing grooves and locks where the base of the sheet contains locks and rebates in which the side walls of the bottom module are inserted, and the rear wall of the module contains a latch, and the side wall of the plate is equipped with hooks for attaching another module.

The aim of the device according to the present invention is provision of such a nesting habitat for solitary bees that will enable acquisition, without disturbing the natural developmental processes, of bee cocoons or larvae with the same parameters as are acquired in the natural process as well as a reduction in workload and costs related to breeding these bees, without an adverse effect on the insects themselves. Due to the high effectiveness of pollination of cultivated plants, it is of high importance to implement easy and effective ways of breeding in materials that provide alternative forms of natural nests. In addition, the aim of the solution is to provide breeding combs containing nesting tunnels of varying sizes, adapted to the size of the bees. Furthermore, the aim of the solution according to the present invention is to optimise the thermal environment inside the hive and to allow moisture to drain from inside the nesting tunnel. At the same time, the aim of the invention is to optimise the structure of the sheet to enable mechanical handling of the breeding comb.

A breeding comb for solitary bees according to the invention, comprised of nesting sheets stacked one on top of another and containing grooves forming nesting tunnels and locks with a closed hole on one side, is characterised in that each of the stacked nesting sheets contains:
grooves forming nesting tunnels of different sizes;
over the side edges of the sheet and on the back wall, there are lugs that enable placing over the grooves of the membrane;
in the lower part of the base of the nesting sheet, there are locks in the form of lugs that make it possible to press the diaphragm against the locks of the lower sheet thus dosing the nesting tunnels; the same also form pass-through channels;
in the front part of the sheet there are slide guides in place, adjusted so as to enable sliding the shutter into place;
on the outer sides of the sheets, there are interfaces to allow mechanical separation of the sheets,

where the individual nesting sheets are separated from each other by a membrane while at the front part of the breeding comb there is a shutter in place containing inlet holes of varying sizes, corresponding to the size of the nesting tunnels, and holes corresponding to the size of the pass-through channels.

It is advantageous if the membrane is made of a natural material.

It is advantageous if the membrane is made of cardboard.

It is also advantageous if the shutter is made of a natural material, advantageously of cardboard or plywood.

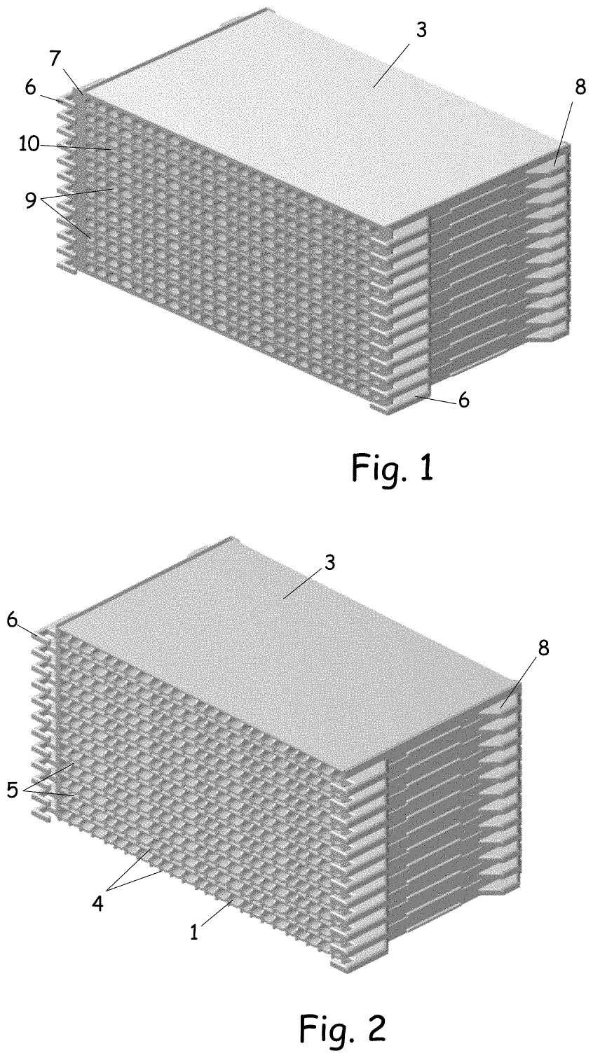

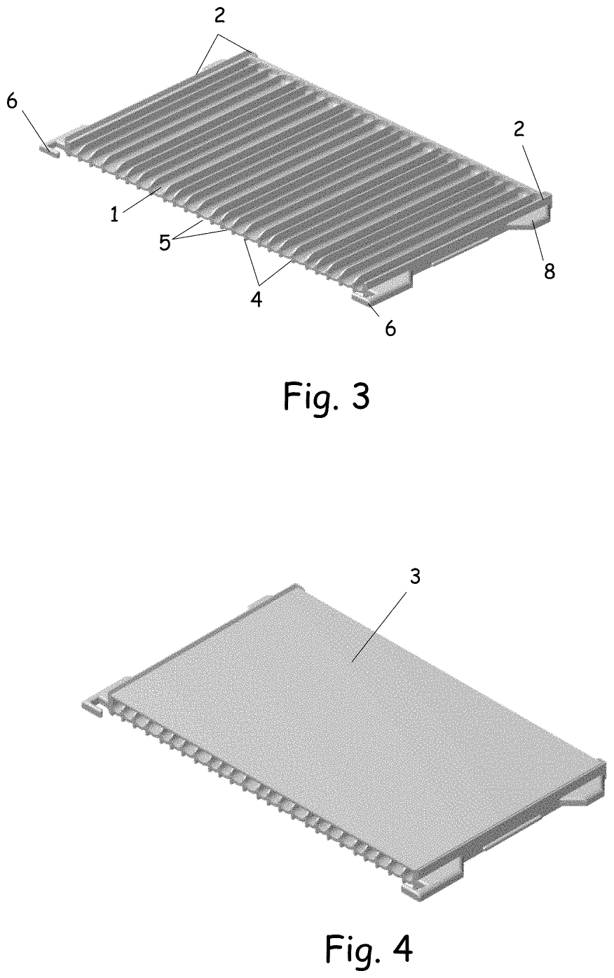

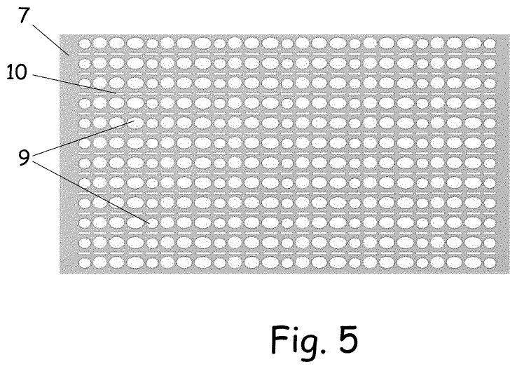

The invention is shown in drawings where FIG. 1 shows an axonometric view of the breeding comb with the shutter in place, FIG. 2 shows an axonometric view of the breeding comb without the shutter, FIG. 3 shows an axonornetric view of the nesting sheet, FIG. 4 shows an axonometric view of the nesting sheet with the membrane in place and FIG. 5 the shutter of the breeding comb.

In a non-limiting embodiment, the breeding comb for solitary bees is built of nesting sheets stacked one on top of another. Each of the nesting sheets contains grooves forming nesting tunnels 1 of different sizes, allowing for better adjustment of the channels to bees of different sizes. On the side edges of the sheet and on the back wall there are lugs 2 which allow placing membrane 3 over the grooves 1 while in the lower part of the base of the nesting sheet there are locks in the form of lugs 4 which allow pressing the membrane 3 to the nesting sheet situated underneath and thus closing nesting tunnels 1 and at the same time forming pass-through channels 5 which allow draining excess moisture and stabilise temperature inside the nesting tunnel 1. In the front part of the nesting sheet there are slide guides 6 adjusted so as to enable sliding shutter 7 while on the external sides of the nesting sheet there are interfaces 8 allowing for mechanical separation of the individual sheets.

The individual nesting sheets are separated by a membrane 3 and in the front part of the comb there is a shutter 7 with inlet holes 9 which gives the whole a more natural character and reduces the temperature inside the breeding comb where inlet holes 9 are of different sizes corresponding to the sizes of nesting channels 1 so that bees can more easily find their nesting tunnel 1. Shutter 7 also contains holes 10 corresponding to the size of the pass-through channels 5.

In a non-limiting embodiment, membrane 3 is made of a natural material (cardboard) and shutter 7 is made of a natural material, advantageously of cardboard or plywood.

1. Breeding comb for solitary bees comprised of nesting sheets stacked one on top of another and containing grooves forming nesting tunnels and locks with a dosed hole on one side, characterised in that each of the stacked nesting sheets contains:
grooves forming nesting tunnels (1) of different sizes;
lugs (2) placed over the side edges of the sheet and on the back wall;
locks in the form of lugs (4) in the lower part of the base of the nesting sheet where the said lugs form pass-through channels (5);
slide guides (6) placed in the front part of the sheet;
interfaces (8) placed on outer sides of the sheets;
where the individual nesting sheets are separated from each other by a membrane (3) while at the front part of the breeding comb there is a shutter (7) in place containing inlet holes (9) of varying sizes corresponding to the size of the nesting tunnels (1) and holes (10) corresponding to the size of the pass-through channels (5),

2. The breeding comb according to claim 1 characterised in that the membrane (3) is made of a natural material.

3. The breeding comb according to claim 2 characterised in that the membrane (3) is made of cardboard.

4. The breeding comb according to claim 1 characterised in that the shutter (7) is made of a natural material, advantageously of cardboard or plywood.
